(19) United States
(12) Patent Application Publication
(10) Pub. No.: US 2010/0256485 A1
(43) Pub. Date: Oct. 7, 2010

(54) MICROWAVE CARDIOPULMONARY SENSING METHOD AND APPARATUS

(76) Inventors: Jon Gordan Ables, New South Wales (AU); Suzan Pollicino, New South Wales (AU); Cong Nhin Huynh, New South Wales (AU); Robert Douglas Shaw, New South Wales (AU); Kamil Unver, New South Wales (AU)

Correspondence Address: BRINKS HOFER GILSON & LIONE, P.O. BOX 10395, CHICAGO, IL, 60610, US

(21) Appl. No.: 12/296,857

(22) PCT Filed: Apr. 12, 2007
(86) PCT No.: PCT/AU07/00486

§ 371 Date: Apr. 22, 2010

(30) Foreign Application Priority Data
Apr. 13, 2006 (AU) 2006901967

(51) Int. Cl. A61B 6/00

(52) U.S. Cl. 600/430

(57) ABSTRACT

A method and system of monitoring changes in a body, the method comprising the steps of: (a) emitting microwave radiation from a set of spaced apart transmitters placed adjacent the body; (b) separately receiving a radiation pattern from the transmitters via at least one receiver; (c) analysing the differences between the separately received radiation patterns to determine changes in the body.

### FIELD OF THE INVENTION

The present invention relates to the field of microwave sensing of body organ activity and, in particular, discloses a method and apparatus for sensing organ activity within humans or animals.

### BACKGROUND OF THE INVENTION

Various methods are known for measuring or monitoring organ activity within the human or animal body. In particular, heart and lung monitoring methods are known.

Previous non-imaging and non-invasive approaches have been based on radar principles, pressure sensors (plethysmographs), variants of electrocardiography (ECG) or phonocardiography. The radar based methods that have been reported (both pulsed and continuous wave) perform poorly—and there is thought to be no commercial heart monitoring devices using radar. The plethysmograph works well but must be clipped or taped to the body (fingertip, earlobe, forehead etc.) and gives only the heart rate, although it can be extended to provide oximetry. ECG methods needs ohmic “touch” contact with the skin at multiple sites. Good ECG tracings can be excellent diagnostically but require expert interpretation. Some work on capacitive, non-ohmic electrodes has been reported but these suffer from variability and a lack of robustness. The best are not actually non-contacting since the insulation (e.g., butyl rubber) that separates the electrode from the skin must touch the skin. Also all ECG methods are subject to electrical noise signals produced by the skeletonal muscles but give almost no information on lung action. Phonocardiograms are useful, but provide only limited quantitative information.

### SUMMARY OF THE INVENTION

An objective of the present invention is to provide a non-invasive body monitoring capability using microwave sensing of sub-surface or otherwise hidden organs within the body, distinguished by their spatial inhomogeneities and/or temporal variation, which provides information not presented by current devices. Although applicable to other uses, a method and apparatus is herein disclosed which is particularly suitable for non-invasive sensing of organ activity within humans or animals.

Disclosed herein is a method and apparatus for monitoring changes in a body, the method comprising the steps of: (a) projecting radiation through the body along at least two closely spaced paths; (b) analysing the differences in the received responses of the radiation patterns after projection along the at least two closely space paths to determine changes in portions of said body.

The spaced apart transmitters can emit radiation in a time-multiplexed manner for reception by at least one receiver in a synchronously time-multiplexed manner. The number of transmitters can be two and the number of receivers can be one. Due to the duality between transmitters and receivers, in any configuration the roles of transmitters and receivers can be reversed.

### BRIEF DESCRIPTION OF THE DRAWINGS

Preferred forms of the present invention will now be described referencing the accompanying drawing in which:

FIG. 1 illustrates schematically the arrangement of the preferred embodiment;

FIG. 2 illustrates the electronic circuit of a preferred embodiment in more detail;

FIG. 3 shows a flow diagram for an embodiment of a method of monitoring changes in a body;

FIG. 4 shows a flow diagram for another embodiment of a method of monitoring changes in a body; and

FIG. 5 shows a flow diagram for another embodiment of a method of monitoring changes in a body.

### DESCRIPTION PREFERRED AND OTHER EMBODIMENTS

In the preferred embodiment, there is provided a novel and inventive method of utilising the simple non-coherent detection of volume-scattered microwaves to provide for organ monitoring capabilities. The preferred embodiments utilise a switched comparison method employing a lock-in amplifier for detection of the differences in the power of the scattered microwave radiation from within two or more volumes within the body.

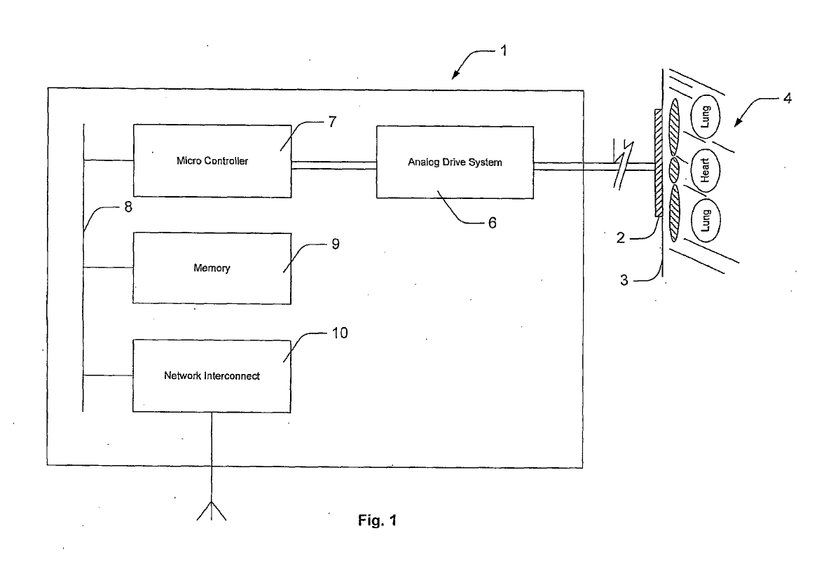

Turning initially to FIG. 1, there is illustrated schematically one form of arrangement of the preferred embodiment 1, wherein a series of microwave transmission and reception antennaes 2 are placed alongside a human body 3 adjacent to the heart and lung system 4. The antennae system 2 is driven by an analog drive system 6 under the control of a micro controller 7. The micro controller 7 provides for digital processing capabilities in the device 1 and is interconnected via bus 8 to memory 9 and external network connection devices 10.

It will be apparent from those skilled in the art that other arrangements to that disclosed schematically in FIG. 1 are possible however the arrangement of FIG. 1 is designed to provide for a wearable portable battery powered device that can be radio linked to remote login and networking devices. The network interconnect 10 can provide standard wireless network interconnections such as 802.11 networking capability. Additionally, the device 1 may optionally have its own user interface. Alternatively, a non-optimal form may include tethering the monitoring capability to a base station.

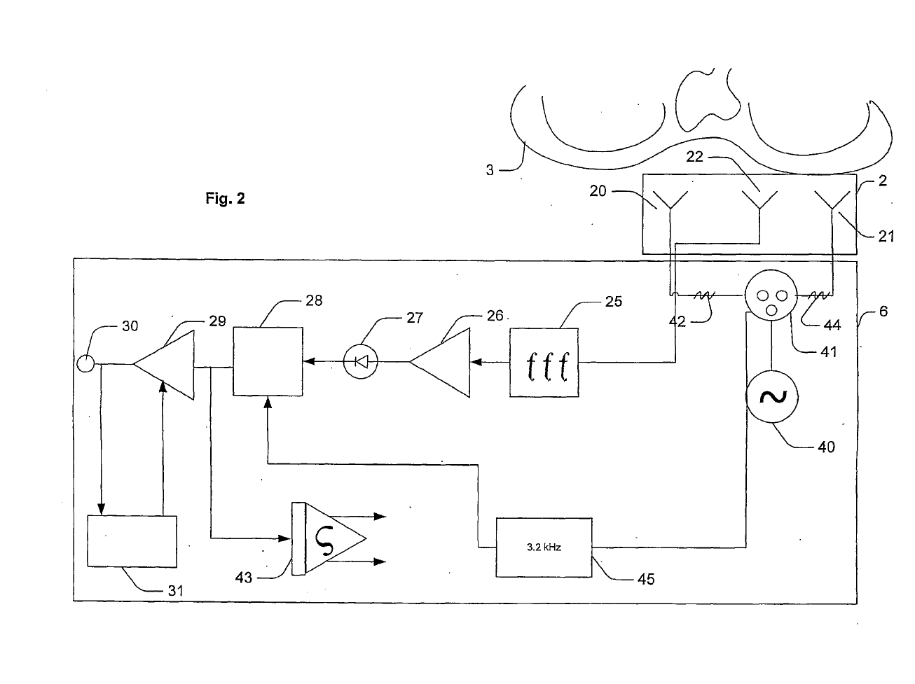

Turning now to FIG. 2, there is illustrated the schematic arrangement of a preferred embodiment in more detail. Three microwave antennas including two transmission antennas 20, 21 and one receiver antenna 22 are provided for placement proximal to the body to be measured. The antenna forms may include near isotropic, sub-wavelength sized “elemental” forms spaced apart by sub-wavelength distances. These antennas can be separately packaged in a tethered module. Geometric symmetry in the placement of the antennas simplifies post-processing but is not mandatory.

The receiver antenna 22 is connected to a processing train that includes a first band pass filter 25, a logarithmic amplifier 26, a power detector 27 and a lock-in amplifier 28. This lock-in amplifier having a phase sensitive detector. The output is low-pass filtered and further amplified 29 before output 30. The output is automatic gain controlled by AGC servo 31.

The two outer transmitters 20, 21 are driven in turn by a continuous-wave microwave oscillator 40. The output signal is switched from one antenna to the other via a single-pole, double-throw (SPDT) RF switch 41 so that the microwave power is directed to one or other of the transmitting antennas 20, 21 in turn. The position of the switch 41 is electronically controlled. The output power delivered to each of the transmitters is electronically controlled by a balance servo 43.

The switching between antennas is electronically controlled by a clock signal 45 which can comprise a stable audio-frequency reference oscillator. The reference also controls the lock-in sample amplifier 28. Hence, the receiver antenna 22 is alternately presented with scattered radiation from the vicinity of each of the two outer antennas, switched at the clock rate.

The same clock signal 45 forms the switching reference for the lock-in amplifier 28. The output of the lock-in amplifier 28 will be proportional to the difference between the decibel measure of the observed scattered powers from the two outer antennas. The difference signal is further amplified in the low pass amplifier 29 which provides amplification from DC to about 35 hertz and which contains an AGC servo 31 to regulate the signal amplitude.

Broader band output is possible although at very high bandwidths an increase in the clock frequency may be necessary.

The receiver chain 22 to 31 thereby detects small differences in the scattered radiation from the two transmitters 20, 21. The small differences can be sensed even in the presence of large changes that are common to both sides. Such common changes may be the result of breathing, body movement, RF oscillator power level drifts and gain changes in the circuits.

The breathing signals, which tend to be common mode, are best preserved in the sum signal output of the lock-in amplifier (not shown).

The circuit operates best if it is near the balance point where the long-term average of the difference signal is approximately zero. For this reason, an auto-balance servo 43 is included. This adjusts the variable RF attenuators 42, 44 to restore any long-term imbalance that can arise from circuit drift, persistently different tissue samples and misalignment of the antenna system 2 when it is placed near the chest wall.

The analog output 30 may be forwarded to a microcontroller (7 of FIG. 1) where it is converted to a digital signal and logged for analysis. The microcontroller may be connected to any communications network for remote sensing, analysis and logging.

The foregoing describes a preferred form of the present invention in its most simple invocation of having the minimum number (2) of scattering paths. Modification, obvious to those skilled in the art, can be made thereto without departing from the scope of the invention. For example, other numbers of transmitters and receivers may be utilised in various arrays and processing of the received difference signals undertaken so as to provide not just temporal but spatial information on resultant body movements. Some sub-systems, for example the AGC and balance servos and the logarithmic form of the RF amplifier, may not be required in all cases.

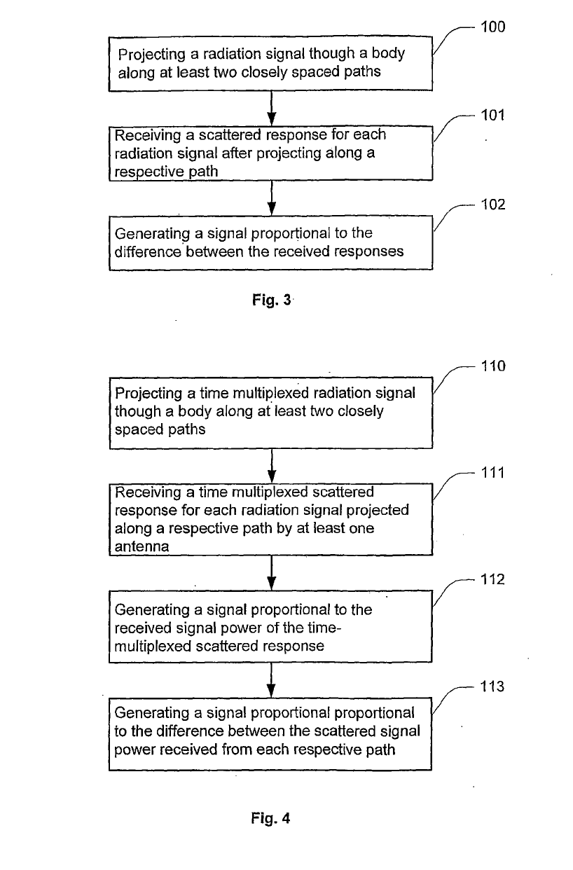

FIG. 3 shows a flow diagram for an embodiment of a method of monitoring changes in a body. In this embodiment the method comprises the steps of:

(a) projecting a radiation signal though a body along at least two closely spaced paths 100;

(b) receiving a scattered response for each radiation signal after projecting along a respective path 101; and

(c) generating a signal proportional to the difference between the received responses 103.

In an embodiment a radiation signal is projected though the body along two paths. These radiation signals may be non-coherent. The difference between the received responses is preferably measured relative to their volume (e.g. received power).

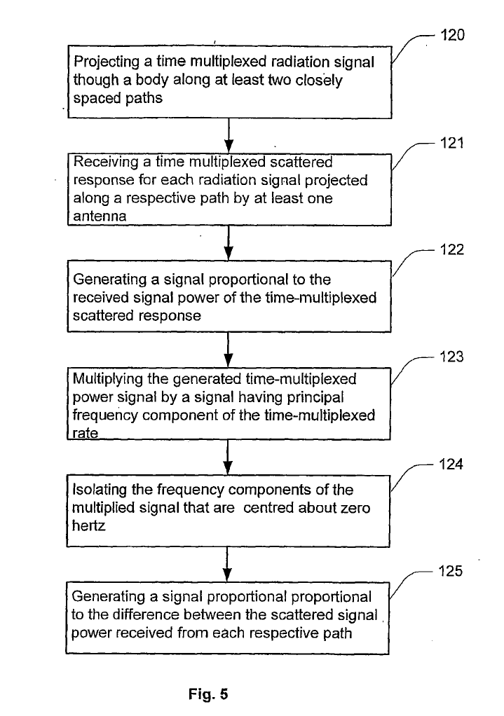

FIG. 4 shows a flow diagram for another embodiment of a method of monitoring changes in a body. In this embodiment the method comprises the steps of:

(a) projecting a time-multiplexed radiation signal though a body along at least two closely spaced paths 110;

(b) receiving a time-multiplexed scattered response for each radiation signal projected along a respective path by at least one receiver 111;

(c) generating a signal proportional to the received signal power of the time-multiplexed scattered response 112; and

(d) generating a signal proportional to the difference between the scattered signal power received from each respective path 113.

In an embodiment radiation signal are projected though the body along two paths. These radiation signals are time-multiplexed, whereby an output signals alternates between one of two transmitters. The received signal is then received by a single receiver, and contains a time-multiplexed signal comprising the scattered signal along the respective path between each transmitter and the receiver. Due to the duality between transmitters and receivers, in any configuration the roles of transmitters and receivers can be reversed.

Preferably a signal proportional to the power of the received signal is generated, also having a time-multiplexed response for each respective path. This generated signal, being time-multiplexed between two independent signals, comprises frequency components centered about the time-multiplexed rate that are proportional to the difference between the two signals.

These frequency components centered about the time-multiplexed rate are selectively measured to produce a signal proportional to the difference between the scattered signal power received from each respective path. In one embodiment this selectively measurement is performed by an analog lock-in amplifier. In another embodiment a processor may perform the function of a lock-in amplifier.

In these embodiments the function of the lock-in amplifier comprises a phase sensitive detector that detects the time-multiplexed switching signal and produces a reference signal having a principal frequency component at the time-multiplexed rate.

The lock-in amplifier then multiplies the reference signal with the time-multiplexed response. This multiplication of the reference signal with the time-multiplexed response produces an output signal that comprises a copy of the difference frequency components, originally centered about time-multiplexed rate, now centered about zero hertz.

In these embodiments the difference component is further isolated by low pass filter. The resulting signal is proportional to the difference between the scattered signal power received from each respective path.

FIG. 5 shows a flow diagram for another embodiment of a method of monitoring changes in a body. In this embodiment the method comprises the steps of:

(a) projecting a time-multiplexed radiation signal though a body along at least two closely spaced paths 120;

(b) receiving a time-multiplexed scattered response for each radiation signal projected along a respective path by at least one antenna 121;

(c) generating a signal proportional to the received signal power of the time-multiplexed scattered response 122;

(d) multiplying the generated time-multiplexed power signal by a signal having principal frequency component of the time-multiplexed rate 123;

(e) isolating the frequency components of the multiplied signal that are centered about zero hertz 124; and

(f) generating a signal proportional to the difference between the scattered signal power received from each respective path 125.

A person skilled in the art would further identify that appropriate portions of the above methods can be similarly performed using either digital or analog techniques. It will be understood that performed in one embodiment the appropriate steps of methods are by a processor (or processors) of a computer system executing instructions (computer-readable code). It will also be understood that the invention is not limited to any particular implementation or programming technique and that the invention may be implemented using any appropriate techniques for implementing the functionality described herein.

Further, a processor may perform additional control and post processing of signals. In alternative embodiments this processor may receive the resulting signal, being proportional to the difference between the scattered signal power received from each respective path, through a communications network via a wired or wireless connection.

### Interpretation

Unless specifically stated otherwise, as apparent from the following discussions, it is appreciated that throughout the specification discussions utilizing terms such as “processing,” “computing,” “calculating,” “determining” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data. A “computer” or a “computing machine” or a “computing platform” may include one or more processors. In alternative embodiments, the one or more processors operate as a standalone device or may be connected, e.g., networked to other processor(s), in a networked deployment, the one or more processors may operate in the capacity of a server or a client machine in server-client network environment, or as a peer machine in a peer-to-peer or distributed network environment.

In the context of this document, the term “wireless” and its derivatives may be used to describe circuits, devices, systems, methods, techniques, communications channels. The term does not imply that the associated devices do not contain any wires.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. Functionality may be added or deleted from the block diagrams and operations may be interchanged among functional blocks. Steps may be added or deleted to methods described within the scope of the present invention.

1. A method of monitoring changes in a body, the method comprising the steps of:
a) projecting radiation through the body along at least two closely spaced paths;
b) analysing differences in received responses of radiation patterns after projection along the at least two closely space paths to determine changes in portions of the body.

2. A method as claimed in claim 1 wherein the step (a) further comprises emitting the radiation from at least two spaced apart transmitters.

3. A method as claimed in claim 1 wherein the step (a) further comprises receiving the radiation responses utilising at least two spaced apart receivers.

4. A method as claimed in claim 2 wherein the spaced apart transmitters emit the radiation in a time-multiplexed manner for reception by at least one receiver in a time-multiplexed manner.

5. A method as claimed in claim 2 wherein one of the transmitters is attached to a wall of the body.

6. A method as claimed in claim 2 wherein a number of transmitters is two.

7. A method of monitoring changes in a body, the method comprising the steps of:
a) emitting microwave radiation from a set of spaced apart transmitters placed adjacent to the body;
b) separately receiving a radiation pattern from the transmitters via at least one receiver; and
c) analysing differences between separately received radiation patterns to determine changes in the body.

8. A method of monitoring changes in a body, the method comprising the steps of:
a) projecting a time-multiplexed radiation signal though a body along at least two closely spaced paths;
b) receiving a time-multiplexed scattered response for each radiation signal projected along a respective path by at least one receiver;
c) generating a first signal proportional to a received signal power of the time-multiplexed scattered response; and
d) generating a second signal proportional to a difference between a scattered signal power received from each respective path.

9. A method of monitoring changes in a body, the method comprising the steps of:
a) projecting a time-multiplexed radiation signal though a body along at least two closely spaced paths;
b) receiving a time-multiplexed scattered response for each radiation signal projected along a respective path by at least one antenna;
c) generating a first signal proportional to a received signal power of the time-multiplexed scattered response;
d) multiplying the generated time-multiplexed power signal by a second signal having a principal frequency component of a time-multiplexed rate;
e) isolating frequency components of the multiplied signal that are centered about zero hertz; and
f) generating a third signal proportional to a difference between a scattered signal power received from each respective path.

10. A method as claimed in claim 1 whereby the generation of the difference in received responses is effected by a lock-in amplifier (also known as phase-sensitive or synchronous detection) technique employing a common clock for time-multiplexing of radiation transmitters and receivers.

11. A method as claimed in claim 1 wherein values and/or differences of the received responses are digitized and passed to a microcomputer system for storage and further analysis.

12. A method as claimed in claim 1 wherein the microcomputer system controls any or all parameters of the said transmitters, receivers and lock-in amplifiers.

13. A method as claimed in claim 1 wherein the said microcomputer system passes data from a sensor system to a communications network via a wired or wireless connection.

14. (canceled)

15. A body change sensing system comprising:
a series of transmitters and receivers for projecting radiation along at least two paths within a body and receiving reflected radiation from each of the paths; and
a processing means for processing separately received reflected radiation from said paths so as to determine difference therein.

16. A system as claimed in claim 15 wherein the radiation along each of said paths is emitted in a time-multiplexed manner.

17. A system as claimed in claim 15 wherein the number of transmitters is two and the number of receivers is one.

18. A system as claimed in claim 15 whereby the formation of the differences in received reflected radiation is effected by a lock-in amplifier (also known as phase-sensitive or synchronous detection) technique employing a common clock for the time-multiplexing of the transmitters and the receivers.

19. A system as claimed in claim 15 wherein the values and/or the differences of the received reflected radiation are digitized and passed to a microcomputer system for storage and further analysis.

20. A system as claimed in claim 18 wherein a microcomputer system controls any or all parameters of the transmitters, the receivers and the lock-in amplifiers.

21. A system as claimed in claim 15 wherein a microcomputer system passes data from a sensor system to a communications network via a wired or wireless connection.

22. (canceled)

23. A method as claimed in claim 3 wherein one of the receivers is attached to a wall of the body.

24. A method as claimed in claim 3 wherein a number of receivers is one.
